(19) United States
(12) Patent Application Publication
(10) Pub. No.: US 2020/0009289 A1
(43) Pub. Date: Jan. 9, 2020

(54) COMPOSITIONS AND METHODS FOR PROMOTING HEMOSTASIS

(71) Applicant: LOMA LINDA UNIVERSITY HEALTH, Loma Linda, CA (US)

(72) Inventors: Mahmoud TORABINEJAD, Loma Linda, CA (US); Richard MASKIEWICZ, Loma Linda, CA (US)

(73) Assignee: LOMA LINDA UNIVERSITY HEALTH, Loma Linda, CA (US)

(21) Appl. No.: 16/490,657

(22) PCT Filed: Mar. 2, 2018
(86) PCT No.: PCT/US2018/020766

§ 371 Date: Sep. 3, 2019

Related U.S. Application Data
(60) Provisional application No. 62/466,507, filed on Mar. 3, 2017.

(51) Int. Cl. A61L 15/42; A61L 15/44; A61L 15/26; C08J 9/40; A61L 15/18

(52) U.S. Cl. CPC A61L 15/425; A61L 15/44; A61L 15/26; C08G 18/3206; A61L 15/18; A61L 2300/418; A61L 2400/04; C08J 9/40

(57) ABSTRACT

In one aspect, compositions for promoting hemostasis are provided. In some embodiments, the composition comprises a porous non-fibrous substrate impregnated with a hemostatic agent, wherein the porous non-fibrous substrate comprises a hydrophilic polyurethane foam. Methods of promoting hemostasis in a subject using such compositions, and methods of manufacturing such compositions, are also provided.

### BACKGROUND OF THE INVENTION

The present application claims priority to U.S. Provisional Patent Application No. 62/466,507, filed Mar. 3, 2017, the entire contents of which are incorporated by reference herein.

### BACKGROUND OF THE INVENTION

Hemostasis and acquisition of a dry surgical field are important factors for successful management of an endodontic surgery. Localized hemorrhage control not only enhances visibility and assessment of the root structure, but also ensures the appropriate environment for root-end filling placement and minimizes root-end filling contamination. Thus, adequate hemostasis during endodontic surgery creates a better working environment for the operator.

The primary hemostasis during endodontic surgery is usually achieved by administration of local anesthesia with epinephrine. Local hemostatic agents are good adjuncts for hemostasis after administration of local anesthesia with epinephrine. They provide local hemostasis by controlling bleeding from small blood vessels or capillaries. See, Kim et al., J. Endod, 2006, 32:601-623. Hemostatic agents used in endodontic surgery include epinephrine cotton pellets, ferric sulfate, calcium sulfate, bone wax, collagen-based materials, SURGICEL®, Gelfoam®, aluminum chloride, and HemCon.

Cotton pellets containing racemic epinephrine HCl are commercially available. However, the retention of cotton fibers in the surgical site is a critical concern with the use of epinephrine-impregnated cotton pellets. See, e.g., Ibarrola et al., J. Endodo, 1985, 11:75. Strands of cotton fibers are easily pulled away from the pellets and may be retained in the surgical site. Loose cotton fibers left in the surgical site may affect the root-end seal by becoming trapped between the root-end cavity preparation and the root-end filling material. Additionally, cotton fibers may serve as foreign bodies in the surgical site and cause impaired or delayed wound healing. Gutmann et al., Surgical Endodontics, Ishiyaku EuroAmerica, St. Louis, Mo. (1994).

Multiple case reports have warned about the undesirable tissue reaction induced by retained cotton fibers. For example, Sexton et al. (Skeletal Radiol, 1981, 7:211-213) report an intensive foreign body reaction from a retained cotton sponge, which developed a large soft tissue mass in the femur. Kalbermatten et al. (Skeletal Radiol., 2001, 30:415-417) also report a pseudo-tumor of the femur, which was induced by remnants of the cotton sponge. Yet another report presented an intracranial foreign body granuloma caused by fine cotton fibers left during a previous brain tumor operation. Nakayama et al., No Shinkei Geka., 1994, 22:1081-1084.

Accordingly, there remains a need for compositions that promote hemostasis, both in endodontic surgery and in other applications.

### BRIEF SUMMARY OF THE INVENTION

In one aspect, compositions for promoting hemostasis are provided. In some embodiments, the compositions comprise a porous non-fibrous substrate impregnated with a hemostatic agent, wherein the porous non-fibrous substrate comprises a hydrophilic polyurethane foam. In some embodiments, the compositions comprise a porous non-fibrous substrate impregnated with a hemostatic agent, wherein the porous non-fibrous substrate comprises a hydrophilic blend of polyurethane and polyethylene glycol or polyethylene oxide.

In some embodiments, the hemostatic agent is a vasoconstrictor or a chemical cauterizing agent. In some embodiments, the hemostatic agent is a vasoconstrictor. In some embodiments, the vasoconstrictor is epinephrine. In some embodiments, the hemostatic agent is a chemical cauterizing agent. In some embodiments, the chemical cauterizing agent is ferric sulfate or calcium sulfate.

In some embodiments, the composition comprises epinephrine in an amount from about 0.45 mg to about 0.75 mg (e.g., per device). In some embodiments, the composition comprises epinephrine in an amount of about 0.45 mg, about 0.5 mg, about 0.55 mg, about 0.6 mg, about 0.65 mg, about 0.7 mg, or about 0.75 mg.

In some embodiments, the composition is in the form of a pellet. In some embodiments, the pellet has dimensions of about 3-5 mm×3-5 mm×3-5 mm.

In some embodiments, the hydrophilic polyurethane foam makes up at least 80% of the weight of the composition. In some embodiments, the hydrophilic polyurethane foam has a density of about 2 pounds per cubic foot to about 5 pounds per cubic foot. In some embodiments, the hydrophilic polyurethane foam has a density of about 3.8 pounds per cubic foot. In some embodiments, the hydrophilic polyurethane foam absorbs an aqueous solution (e.g., a surfactant-free and/or organic co-solvent-free aqueous solution) that is applied to the surface of the foam within about 5 seconds of application. In some embodiments, the hydrophilic polyurethane foam is open celled.

In another aspect, methods of manufacturing compositions for promoting hemostasis are provided. In some embodiments, the method comprises:
applying an aqueous solution comprising a hemostatic agent onto a surface of a porous non-fibrous substrate comprising a hydrophilic polyurethane foam or a hydrophilic polyurethane blend, wherein the aqueous solution is absorbed by the substrate.

In some embodiments, the aqueous solution comprising the hemostatic agent is completely absorbed by the substrate. In some embodiments, the aqueous solution comprising the hemostatic agent is completely absorbed by the substrate within 5 seconds of applying the aqueous solution.

In some embodiments, the hemostatic agent is a vasoconstrictor or a chemical cauterizing agent. In some embodiments, the hemostatic agent is a vasoconstrictor. In some embodiments, the vasoconstrictor is epinephrine. In some embodiments, the hemostatic agent is a chemical cauterizing agent. In some embodiments, the chemical cauterizing agent is ferric sulfate or calcium sulfate. In some embodiments, the aqueous solution further comprises an antioxidant. In some embodiments, the antioxidant is sodium metabisulfate. In some embodiments, the aqueous solution comprises epinephrine in an amount from about 5 mg/mL to about 15 mg/mL and further comprises sodium metabisulfate in an amount from about 0.75 mg/mL to about 1.25 mg/mL.

In some embodiments, the hydrophilic polyurethane or polyurethane blend foam makes up at least 80% of the weight of the non-fibrous substrate. In some embodiments, the hydrophilic polyurethane or polyurethane blend foam has a density of about 2 pounds per cubic foot to about 5 pounds per cubic foot. In some embodiments, the hydrophilic polyurethane foam has a density of about 3.8 pounds per cubic foot. In some embodiments, the hydrophilic polyurethane or polyurethane blend foam absorbs an aqueous solution (e.g., a surfactant-free and/or organic co-solvent-free aqueous solution) that is applied to the surface of the foam within about 5 seconds of application.

In some embodiments, the porous non-fibrous substrate is a hydrophilic polyurethane or polyurethane blend foam sheet having a thickness of about 3 mm to about 5 mm. In some embodiments, the applying step comprises applying to the surface of the foam sheet an aqueous solution comprising epinephrine in an amount from about 9 mg/mL to about 10 mg/mL and sodium metabisulfate in an amount from about 0.75 mg/mL to about 1.25 mg/mL, wherein the aqueous solution is applied to the surface of the foam sheet in an amount from about 190 μL to about 325 μL square centimeter of the foam sheet.

In some embodiments, after the applying step, the method further comprises removing essentially all water from the porous non-fibrous substrate. In some embodiments, the water is removed by air drying. In some embodiments, the water is removed by lyophilization.

In yet another aspect, methods of promoting hemostasis in a subject in need thereof are provided. In some embodiments, the method comprises applying a composition as described herein (e.g., a composition comprising a porous non-fibrous substrate impregnated with a hemostatic agent, wherein the porous non-fibrous substrate comprises a hydrophilic polyurethane foam or a blend comprising polyurethane and polyethylene glycol or polyethylene oxide) to an active bleeding site in the subject. In some embodiments, wherein the active bleeding site is a surgical site. In some embodiments, the active bleeding site is an endodontic surgical site. In some embodiments, the active bleeding site is a wound. In some embodiments, the active bleeding site is a nosebleed.

### BRIEF DESCRIPTION OF THE DRAWINGS

FIG. 1. Hemostatic efficacy for negative control group (no application of hemostatic agent), polyurethane foam with epinephrine group, positive control group (bone wax), and Racellet #3 group.

FIG. 2A-D. Representative histological images from (A) negative control group with no application of hemostatic agent, (B) positive control group (bone wax), (C) polyurethane foam with epinephrine group, and (D) Racellet #3 group.

FIG. 3. Degree of foreign body reaction elicited in the negative control (NC) group (no application of hemostatic agent), positive control (PC) group (bone wax), polyurethane foam with epinephrine group, and Racellet #3 group.

### DETAILED DESCRIPTION OF THE INVENTION

### I. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

As used herein, “hemostasis” refers to process of stopping bleeding and/or promoting clotting of a damaged or injured blood vessel or capillary, such as but not limited to an arteriolar, venous, or capillary vessel.

As used herein, a “hemostatic agent” refers to a substance (e.g., a chemical, a biological product, or a biologically derived product) that promotes hemostasis. In some embodiments, a hemostatic agent promotes hemostasis by promoting local vasoconstriction. In some embodiments, a hemostatic agent promotes hemostasis by promoting blood coagulation. In some embodiments, a hemostatic agent is a vasoconstrictor or a chemical cauterizing agent.

As used herein, the term “non-fibrous substrate” refers to a substrate that is composed of non-fibrous materials. In some embodiments, a non-fibrous substrate is a substrate that does not shed or release fibers, e.g., upon removal from an active bleeding site into which the substrate has placed.

The term “impregnated,” as used with reference to a “substrate impregnated with a hemostatic agent,” refers to the absorption of a hemostatic agent (e.g., a hemostatic agent in an aqueous solution) into a substrate (e.g., a substrate comprising an open cell hydrophilic polyurethane foam).

A “subject” is a mammal, in some embodiments, a human. Mammals can also include, but are not limited to, primates (e.g., monkeys), cows, pigs, horses, dogs, cats, mice, rats, and guinea pigs.

### II. Compositions for Promoting Hemostasis

In one aspect, compositions for promoting hemostasis are provided. In some embodiments, the composition comprises a porous non-fibrous substrate impregnated with a hemostatic agent, wherein the porous non-fibrous substrate comprises a hydrophilic polymeric substance. In some embodiments, the composition comprises a porous non-fibrous substrate impregnated with a hemostatic agent, wherein the porous non-fibrous substrate comprises a hydrophilic polyurethane foam. In some embodiments, the composition comprises a porous non-fibrous substrate impregnated with a hemostatic agent, wherein the porous non-fibrous substrate comprises a hydrophilic blend of polyurethane and a polyethylene oxide or a polyethylene glycol. In some embodiments, the porous non-fibrous substrate does not contain any human or animal components. In some embodiments, the porous non-fibrous substrate is synthetic.

### Hydrophilic Polyurethane Foam

In some embodiments, the porous non-fibrous substrate comprises hydrophilic polyurethane foam. As used herein, “polyurethane” refers to a polymer in which the repeating unit contains a urethane moiety. In some embodiments, the polyurethane comprises a combination of “hard” and “soft” segments. The use of a combination of hard and soft segments can impart to the polyurethane certain physical properties, such as flexibility and mechanical strength. As a non-limiting example, in some embodiments, the polyurethane comprises soft segments of DL-lactide and E-caprolactone and hard segments synthesized from butanediol and 1,4-butanediisocyanate. See, e.g., van Minnen et al., J Biomed Mater Res A, 2006, 76:377-385.

In some embodiments, the hydrophilic polyurethane foam is a medical grade polyurethane foam that is able to absorb an aqueous solution that is applied onto the surface of the foam. In some embodiments, the hydrophilic polyurethane foam is open celled (i.e., comprises interconnected cells). In some embodiments, the hydrophilic polyurethane foam has a density of about 2 pounds per cubic foot to about 5 pounds per cubic foot, e.g., about 2, 2.5, 3, 3.5, 4, 4.5, or 5 pounds per cubic foot. In some embodiments, the hydrophilic polyurethane foam has a density of about 3.8 pounds per cubic foot.

In some embodiments, the hydrophilic polyurethane foam is highly absorbent. In some embodiments, the hydrophilic polyurethane foam imbibes or absorbs an aqueous solution (e.g., a surfactant-free and/or organic co-solvent-free aqueous solution) that is applied to the surface of the foam within about 30 seconds of application, e.g., within about 15 seconds of application, within about 10 seconds of application, or within about 5 seconds of application. In some embodiments, the hydrophilic polyurethane foam completely absorbs (becomes saturated with) an aqueous solution that is applied to the surface of the foam within about 30 seconds of application, e.g., within about 15 seconds of application, within about 10 seconds of application, or within about 5 seconds of application. In some embodiments, the hydrophilic polyurethane foam can absorb up to 10 times its weight in fluids, e.g., up to 15 times its weight in fluids, up to 20 times its weight in fluids, or up to 30 times its weight in fluids.

In some embodiments, the hydrophilic polyurethane foam is commercially available. Commercially available foams include, e.g., Capu-Cell™ (Foam Sciences, Buffalo, N.Y.), Hydrasorb® (Carwild Corp., New London, Conn.), ResQFoam (Arsenal Medical, Inc., Watertown, Mass.), Luofucon foam dressing (Foryou Medical Devices Co., Ltd., Guangdong, China), and Allevyn hydrocellular polyurethane dressing (Smith & Nephew, Inc., Andover, Mass.). In some embodiments, the hydrophilic polyurethane foam is a foam disclosed in WO 2013/155254 or U.S. Pat. No. 5,650,450. In some embodiments, the hydrophilic polyurethane foam is a biodegradable polyurethane. See, e.g., van Minnen et al., J Biomed Mater Res A, 2006, 76:377-385.

### Hydrophilic Polyurethane Blend

In some embodiments, the porous non-fibrous substrate comprises a hydrophilic blend of polyurethane and a polyethylene oxide or a polyethylene glycol. In some embodiments, the blend comprises polyurethane and a polyethylene oxide polymer. In some embodiments, the blend comprises polyurethane and a polyethylene glycol polymer. In some embodiments, the blend comprises polyurethane, polyethylene oxide, and polyethylene glycol.

In some embodiments, the blend comprises polyurethane and a polyethylene glycol polymer. In some embodiments, the polyethylene glycol has an average molecular weight of at least 300 g/mol, at least 400 g/mol, at least 600 g/mol, at least 1,000 g/mol, at least 1,500 g/mol, at least 2,000 g/mol, at least 3,000 g/mol, at least 4,000 g/mol, at least 6,000 g/mol, at least 8,000 g/mol, at least 10,000 g/mol, at least 20,000 g/mol, or at least 35,000 g/mol. In some embodiments, the polyethylene glycol has an average molecular weight of about 300 g/mol, 400 g/mol, 600 g/mol, 1,000 g/mol, 1,500 g/mol, 2,000 g/mol, 3,000 g/mol, 4,000 g/mol, 6,000 g/mol, 8,000 g/mol, 10,000 g/mol, 20,000 g/mol, or 35,000 g/mol. In some embodiments, the blend comprises polyurethane and a polyethylene glycol polymer having an average molecular weight of about 20,000 g/mol.

In some embodiments, the blend comprises polyurethane and a polyethylene oxide polymer. In some embodiments, the polyethylene oxide has an average molecular weight of at least 100,000 g/mol, at least 200,000 g/mol, at least 300,000 g/mol, at least 400,000 g/mol, or at least 600,000 g/mol. In some embodiments, the polyethylene glycol has an average molecular weight of about 100,000 g/mol, 200,000 g/mol, 300,000 g/mol, 400,000 g/mol, or 600,000 g/mol.

In some embodiments, the blend comprises a polyurethane as described above. In some embodiments, the blend comprises polyurethane block copolymers comprising hard and soft segments. As a non-limiting example, in some embodiments, the blend comprises a polyurethane that comprises soft segments of DL-lactide and E-caprolactone and hard segments synthesized from butanediol and 1,4-butanedlisocyanate. See, e.g., van Minnen et al., J Biomed Mater Res A, 2006, 76:377-385.

In some embodiments, the blend comprises polyurethane in an amount from about 40% to about 90% and polyethylene oxide or polyethylene glycol in an amount from about 10% to about 60%. In some embodiments, the blend comprises polyurethane in an amount from about 40% to about 70% and polyethylene oxide or polyethylene glycol in an amount from about 30% to about 60%. In some embodiments, the blend comprises polyethylene oxide or polyethylene glycol in an amount up to 60%, up to about 55%, up to about 50%, up to about 45%, up to about 40%, up to about 35%, up to about 30%, up to about 25%, or up to about 20%. In some embodiments, the blend has a polyethylene oxide or polyethylene glycol content of about 10%, about 15%, about 20%, about 25%, about 30%, about 35%, about 40%, about 45%, about 50%, about 55%, or about 60%.

In some embodiments, the porous non-fibrous substrate comprises a hydrophilic polyurethane blend as disclosed in US 2012/0144592.

In some embodiments, the hydrophilic polyurethane blend has a density of about 2 pounds per cubic foot to about 5 pounds per cubic foot, e.g., about 2, 2.5, 3, 3.5, 4, 4.5, or 5 pounds per cubic foot. In some embodiments, the hydrophilic polyurethane blend is highly absorbent. For example, in some embodiments, the hydrophilic polyurethane blend imbibes or absorbs an aqueous solution (e.g., a surfactant-free and/or organic co-solvent-free aqueous solution) that is applied to the surface of the hydrophilic polyurethane blend within about 30 seconds of application, e.g., within about 15 seconds of application, within about 10 seconds of application, or within about 5 seconds of application.

### Hemostatic Agents

In some embodiments, the hemostatic agent is a vasoconstrictor. Suitable vasoconstrictors include, but are not limited to, epinephrine, norepinephrine, and levonordefrin. In some embodiments, the hemostatic agent is epinephrine (e.g., racemic epinephrine). In some embodiments, the epinephrine is in a salt form, e.g., epinephrine hydrochloride (HCl). Reference herein to epinephrine also includes a reference to a pharmaceutically acceptable salt of epinephrine unless otherwise indicated or clear from context. In some embodiments, a combination of two or more vasoconstrictors is used.

In some embodiments, the hemostatic agent is a chemical cauterizing agent. Suitable chemical cauterizing agents include, but are not limited to, ferric sulfate, ferrous sulfate, calcium sulfate, aluminum chloride, zinc chloride, and silver nitrate. In some embodiments, a combination of two or more chemical cauterizing agents is used. In some embodiments, a combination of a vasoconstrictor and a chemical cauterizing agent is used.

In some embodiments, the hemostatic agent is epinephrine, and the epinephrine is present in an amount from about 0.3 mg to about 1.5 mg per device (e.g., pellet), e.g., from about 0.45 mg to about 0.75 mg per device, from about 0.5 mg to about 1 mg per device, or from about 0.5 mg to about 1.5 mg per device. In some embodiments, the hemostatic agent is epinephrine, and the epinephrine is present in an amount of about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.1 mg, about 1.2 mg, about 1.3 mg, about 1.4 mg, or about 1.5 mg per device (e.g., pellet).

### Characteristics of Compositions

In some embodiments, the porous non-fibrous substrate makes up at least 80% of the weight of the composition, e.g., at least 85%, at least 90%, or at least 95% of the weight of the composition. In some embodiments, the hydrophilic polyurethane foam or the hydrophilic blend of polyurethane and a polyethylene oxide or a polyethylene glycol makes up at least 80% of the weight of the composition, e.g., at least 85%, at least 90%, or at least 95% of the weight of the composition. In some embodiments, the hydrophilic polyurethane foam or the hydrophilic blend of polyurethane and a polyethylene oxide or a polyethylene glycol makes up less than 100% of the weight of the composition. In some embodiments, the hydrophilic polyurethane foam or the hydrophilic blend of polyurethane and a polyethylene oxide or a polyethylene glycol makes up at least 80% of the weight of the composition but less than 100% of the weight of the composition, e.g., from about 80% to about 99%, or from about 80% to about 90%, of the weight of the composition.

The compositions for promoting hemostasis can have any suitable shape. For example, in some embodiments, the composition is in the shape of a pellet, a cylinder, a cube, a cone, or a disc. In some embodiments, the composition is a device in the form of a pellet, a sheet, a cube, a square, or a patch. In some embodiments, the composition is in the form of a pellet. In some embodiments, the composition is in the form of a sheet or a patch. In some embodiments, the composition has a size of about 2-10 mm×2-10 mm×2-10 mm, e.g., about 3-5 mm×3-5 mm×3-5 mm. In some embodiments, the composition is from about 25 mm³ to about 500 mm³, e.g., from about 25 mm³ to about 200 mm³, or from about 25 mm³ to about 150 mm³. In some embodiments, the composition has an irregular shape, e.g., flakes or irregularly shaped patches, having an average diameter from about 0.5-10 mm, e.g., about 1-4 mm, 2-6 mm, or 3-5 mm.

In some embodiments, the porous non-fibrous substrate is further impregnated with an antioxidant. In some embodiments, the antioxidant is sodium metabisulfite or sodium metabisulfate.

### III. Methods of Manufacturing Hemostasis Compositions

In another aspect, methods of manufacturing compositions for promoting hemostasis are provided. In some embodiments, the method comprises:
providing a porous non-fibrous substrate comprising a hydrophilic polyurethane foam; and
applying an aqueous solution comprising a hemostatic agent onto the surface of the porous non-fibrous substrate, wherein the aqueous solution is absorbed by the substrate.

In some embodiments, the porous non-fibrous substrate comprising the hydrophilic polyurethane foam is highly absorbent. In some embodiments, the aqueous solution comprising the hemostatic agent is completely absorbed by the substrate. In some embodiments, the aqueous solution comprising the hemostatic agent is completely absorbed by the substrate within about 30 seconds of application, e.g., within about 15 seconds of application, within about 10 seconds of application, or within about 5 seconds of application.

In some embodiments, the aqueous solution comprises a hemostatic agent that is a vasoconstrictor or a chemical cauterizing agent. In some embodiments, the hemostatic agent is a vasoconstrictor. In some embodiments, the vasoconstrictor is epinephrine. In some embodiments, the hemostatic agent is a chemical cauterizing agent. In some embodiments, the chemical cauterizing agent is ferric sulfate or calcium sulfate.

In some embodiments, the aqueous solution further comprises an antioxidant. In some embodiments, the antioxidant is sodium metabisulfite or sodium metabisulfate. In some embodiments, the antioxidant (e.g., sodium metabisulfite or sodium metabisulfate) is present in an amount from about 0.1 mg/mL to about 2 mg/mL, e.g., from about 0.5 mg/mL to about 1.5 mg/mL or from about 0.75 mg/mL to about 1.25 mg/mL. In some embodiments, the antioxidant (e.g., sodium metabisulfite or sodium metabisulfate) is present in an amount of about 0.1 mg/mL, about 0.25 mg/mL, about 0.5 mg/mL, about 0.75 mg/mL, about 1 mg/mL, about 1.25 mg/mL, about 1,5 mg/mL, about 1.75 mg/mL, or about 2 mg/mL.

In some embodiments, the aqueous solution comprises epinephrine in an amount from about 5 mg/mL to about 15 mg/mL, e.g., from about 7 mg/mL to about 12 mg/mL or from about 8 mg/mL to about 10 mg/mL. In some embodiments, the aqueous solution comprises epinephrine in an amount of about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12,5 mg/mL, about 13 mg/mL, about 13.5 mg/mL, about 14 mg/mL, about 14.5 mg/mL, or about 15 mg/mL. In some embodiments, the aqueous solution comprises epinephrine in an amount of about 9.5 mg/mL.

In some embodiments, the aqueous solution comprises epinephrine in an amount from about 5 mg/mL to about 15 mg/mL (e.g., from about 7 mg/mL to about 12 mg/mL or from about 8 mg/mL to about 10 mg/mL , e.g., in an amount of about 5 mg/mL, about 5.5 mg/mL, about 6 mg/mL, about 6.5 mg/mL, about 7 mg/mL, about 7.5 mg/mL, about 8 mg/mL , about 8.5 mg/mL, about 9 mg/mL , about 9.5 mg/mL , about 10 mg/mL , about 10.5 mg/mL, about 11 mg/mL, about 11.5 mg/mL, about 12 mg/mL, about 12.5 mg/mL, about 13 mg/mL, about 13.5 mg/mL , about 14 mg/mL, about 14.5 mg/mL, or about 15 mg/mL) and further comprises sodium metabisulfate in an amount from about 0.75 mg/mL to about 1.25 mg/mL. In some embodiments, the aqueous solution comprises epinephrine in an amount of about 9.5 mg/mL and further comprises sodium metabisulfate in an amount of about 1 mg/mL.

In some embodiments, the hydrophilic polyurethane foam is a polyurethane foam or a hydrophilic polyurethane blend foam described in Section II above and/or has one or more characteristics as described in Section II above (e.g., a medical grade polyurethane foam, or a blend comprising polyurethane and polyethylene glycol or polyethylene oxide). In some embodiments, the hydrophilic polyurethane foam is open celled. In some embodiments, the hydrophilic polyurethane foam has a density of about 2 pounds per cubic foot to about 5 pounds per cubic foot. In some embodiments, the hydrophilic polyurethane foam has a density of about 3.8 pounds per cubic foot. In some embodiments, the hydrophilic polyurethane foam is highly absorbent. In some embodiments, the hydrophilic polyurethane foam absorbs an aqueous solution (e.g., a surfactant-free and/or organic co-solvent-free aqueous solution) that is applied to the surface of the foam within about 5 seconds of application. In some embodiments, the hydrophilic polyurethane foam makes up at least 80% of the weight of the non-fibrous substrate.

In some embodiments, the porous non-fibrous substrate is a hydrophilic polyurethane foam sheet. In some embodiments, the foam sheet has a thickness of about 2 mm to about 10 mm, e.g., from about 2 mm to about 5 mm, from about 3 mm to about 8 mm, or 3 mm to about 5 mm.

In some embodiments, the applying step comprises applying to the surface of the porous non-fibrous substrate (e.g., a hydrophilic polyurethane foam sheet) an aqueous solution comprising epinephrine in an amount from about 9 mg/mL to about 10 mg/mL and sodium metabisulfate in an amount from about 0.75 mg/mL to about 1.25 mg/mL, wherein the aqueous solution is applied to the surface of the porous non-fibrous substrate (e.g., foam sheet) in an amount from about 190 μL to about 325 μL per square centimeter. In some embodiments, the aqueous solution comprising the hemostatic agent (e.g., epinephrine) and optional antioxidant is completely absorbed into the porous non-fibrous substrate (e.g., is completely absorbed into the interior of a hydrophilic polyurethane foam sheet).

In some embodiments, after the applying step, the method further comprises removing water from the porous non-fibrous substrate. In some embodiments, after the applying step, the method further comprises removing essentially all water from the porous non-fibrous substrate. As used herein, “removing essentially all water” means that at least 90% of the water is removed from the substrate. In some embodiments, at least 95%, at least 96%, at least 97%, at least 98%, or at least 99% of the water is removed from the substrate.

In some embodiments, removal of water is accomplished by air drying, vacuum drying, heat drying, lyophilization or freeze drying, or a combination thereof. In some embodiments, the water is removed by air drying. In some embodiments, the water is removed by lyophilization.

In some embodiments, the drying is carried out under conditions of low humidity, such as a relative humidity below 60%, below 50%, or below 40%. In some embodiments, the drying is carried out at a constant relative humidity of below 50%, e.g., a constant relative humidity between about 25%-35%. In some embodiments, the drying is carried out under conditions of low temperature, such as below 27° C., below 25° C., or below 20° C. In some embodiments, the drying is carried out under a temperature that is from about 10° to about 25°, e.g., about 10° to about 15° C. In some embodiments, the drying is carried out at atmospheric pressure or reduced pressures, such as below about 200 mm Hg, or below about 50 mm Hg, at temperatures such as about 25° C to about 90° C.

The drying can be carried out for any desired time period that achieves the desired result, e.g., for about 1 to 20 hours, or from about 4 hours to about 10 hours. Drying may also be carried out for shorter or longer periods of time depending on the product specifications.

In some embodiments, a hemostatic composition as described herein is stored for a prolonged period of time, e.g., at least 1 week, at least 2 weeks, at least 3 weeks, at least 4 weeks, or longer, e.g., at least 1, 2, 3, 4, 5, 6, 7, 8, 9, 10, 11, or 12 months. The hemostatic composition can be stored at various temperatures, such as but not limited to ambient room temperature (e.g., from about 23° C. to about 30° C.), refrigerated temperature (e.g., about 4° C.), or frozen temperature (e.g., about −20° to about −80° C). In some embodiments, the hemostatic composition is stable for at least 1, 2, 3, or 4 weeks or for at least 1, 2, 3, 4, 5, 6, 7, 8, 9, 10, 11, or 12 months when stored at 4° C.

### IV. Methods of Using Compositions for Promoting Hemostasis

In another aspect, methods of use for the hemostatic compositions as described herein are provided. In some embodiments, methods of promoting hemostasis in a subject are provided. In some embodiments, methods of controlling bleeding in a subject are provided. In some embodiments, methods of lessening the severity of bleeding in a subject are provided.

In some embodiments, the method comprises applying a composition as described herein (e.g., a porous non-fibrous substrate comprising a hydrophilic polyurethane foam or polyurethane blend foam and impregnated with a hemostatic agent) to an active bleeding site in a subject. In some embodiments, the active bleeding site can be from any dental or medical procedure associated with bleeding. In some embodiments, a composition as described herein is applied at a surgical site. In some embodiments, the active bleeding site is associated with a surgery or procedure such as, but not limited to, an abdominal surgery, biopsy, cranio-maxillofacial surgery, endodontic surgery (e.g., root canal surgery), ENT (ear, nose, or throat) surgery, general surgery, gingival surgery, oral surgery, orthodontic treatment, orthognathic surgery, organ resection, osseous surgery, periodontal surgery, tooth extraction, tumor resection, or vascular surgery. In some embodiments, the active bleeding site is an endodontic surgical site, e.g., a root canal surgery site. In some embodiments, the active bleeding site is a site of a tooth extraction. In some embodiments, the active bleeding site is a periodontal surgery site.

In some embodiments, a composition as described herein (e.g., a porous non-fibrous substrate comprising a hydrophilic polyurethane foam and impregnated with a hemostatic agent) is applied to a wound or a trauma site. In some embodiments, the wound is an injury to the skin and/or subcutaneous tissue. In some embodiments, a composition as described herein is applied to a nosebleed.

In some embodiments, the composition is applied to the active bleeding site for a period of at least about 10 minutes, at least about 15 minutes, at least about 20 minutes, at least about 30 minutes, at least about 45 minutes, or at least about 60 minutes. In some embodiments, the composition is applied to the active bleeding site for a period of at least about 1 hour, at least about 2 hours, at least about 3 hours, at least about 4 hours, at least about 5 hours, at least about 6 hours, at least about 12 hours, at least about 18 hours, at least about 24 hours, or longer. In some embodiments, the composition is applied to the active bleeding site until blood flow at the site has detectably slowed or has ceased.

In some embodiments, the hemostatic composition that is applied at an active bleeding site does not elicit detectable inflammation after the hemostatic composition is removed from the site. In some embodiments, a detectable foreign body reaction after the hemostatic composition is removed from the site. In some embodiments, the presence or absence of detectable inflammation or a detectable foreign body reaction at the site where the hemostatic composition was applied is measured 1 day, 2 days, 3 days, 4 days, 5 days, 6 days, 7 days, 1 week, 2 weeks, 3 weeks, 4 weeks, 5 weeks, 6 weeks, 7 weeks, 8 weeks or longer after the removal of the hemostatic composition from the site. In some embodiments, detectable inflammation and/or a detectable foreign body reaction is measured by visual inspection, by microscopic inspection, or by histological examination as described in the Examples section below.

### V. Kits

In yet another aspect, kits are provided that comprise one or more compositions for promoting hemostasis. In some embodiments, the kit comprises a porous non-fibrous substrate impregnated with a hemostatic agent, wherein the porous non-fibrous substrate comprises a hydrophilic polyurethane foam or polyurethane blend foam (e.g., a composition as described in Section II above).

In some embodiments, the kit comprises a hemostatic composition as described herein in sterilized packaging. In some embodiments, the kit further comprises instructions for use. In some embodiments, the use is for promoting hemostasis in a subject. In some embodiments, the use is for controlling bleeding in a subject. In some embodiments, the use is for lessening the severity of bleeding in a subject.

### VI. EXAMPLES

The following examples are offered to illustrate, but not to limit, the claimed invention.

### Example 1: Hemostatic Efficacy and Biocompatibility of Epinephrine-Impregnated Polyurethane Foam in Osseous Defects

The purpose of this study was to compare the hemostatic efficacy and biocompatibility of epinephrine-impregnated polyurethane (PU) foam cubes with those of epinephrine-impregnated cotton pellets in osseous defects created in guinea pigs.

### Materials and Methods

### I. Preparation of Test Articles

Hydrophilic, high-density, open-cell polyurethane foam (Cape-Cell; Foam Sciences, Buffalo, N.Y.) (3.8 lb/ft3) cubes in equal size of 0.5 cm³ was obtained from a commercial source. All cubes were autoclaved for sterilization. A 2.25% racemic solution of epinephrine was prepared.

A stock solution of 240 mg epinephrine HCl in 25 mL of 1 mg/mL sodium metabisulfate was freshly prepared and sterile filtered immediately prior to application onto individual foam cubes. Using aseptic technique, 0.55 mg of epinephrine was impregnated per foam cube using a sterile pipette tip under a sterile laminar flow hood. This allowed a direct comparison of the foam cubes to RaceHet® #3 (Pascal Company, Inc.), which contains an average of 0.55 mg of epinephrine per pellet. Foam cubes were then placed in 4 mL HPLC vials and allowed to air dry in the laminar flow hood for 2 hours. The vials were then tightly capped and stored at −20° C. until the day of the animal experiment to minimize potential oxidization of epinephrine.

### II. Procedure

A total of 18 male guinea pigs (750-850 g) were included in the experiment. All procedures were carried out according to the guidelines approved by the Research Committee of Loma Linda University. All procedures were performed aseptically.

The animals were sedated with 3% isoflurane gas and anesthetized by an intramuscular injection of 27.5 mg of ketamine hydrochloride (except for the first 4 animals, in which 55 mg of ketamine hydrochloride was used) and 1.75 mg of xylazine (except for the first 4 animals, in which 3.5 mg of xylazine was used). An injection of 1 mL lidocaine with 1:100,000 epinephrine was given as local anesthetic. The submandibular surgical site of each animal was shaved and disinfected with 5% tincture of iodine. A single extra-oral incision of about 1.5 cm in length was made in the midline over the mandible with a #15 blade and the symphysis was located. After raising a tissue flap with a periosteal and exposing the mandibular cortical bone, a standardized osseous defect of 3 mm in diameter and 2 mm in depth was created with a round bur under continuous saline irrigation on each side of the mandible in the triangle of bone located between the incisor and the caudal side of the symphysis joining the two halves of the mandible.

The animals were divided randomly into 2 groups: the control group (6 animals) and the experimental group (12 animals). Each control group animal had a negative control site on one side of the mandible and a positive control site on the contralateral side by randomized assignment. In the negative control site, the amount of bleeding was measured without any application of hemostatic agents after the osseous defect was created. This negative control served to measure normal hemorrhaging from the defect without any hemostatic intervention and to show normal wound healing. The amount of bleeding was measured by blotting with 15 pre-weighed sterile absorbent paper points (Dentsply Tulsa, Okla., USA) for 2 minutes. The 15 sterile absorbent paper points contained in a HPLC vial were pre-weighed together and after the collection of blood, the paper points were transferred back to the same HPLC vial. The vial was immediately capped airtight to prevent any evaporation of the collected blood. Then at the end of the experiment, these paper points with collected blood in the HPLC vial were weighed again to calculate the difference in the weight before and after the blood collection.

In the positive control site, bone wax was applied to the osseous defect for 2 minutes for hemostasis then removed as much as possible. This positive control served to test the capability of the animals to elicit foreign body reaction by using bone wax which is known to cause foreign body reaction and impair healing when used as a local hemostat. See, e.g., Solheim et al., J Biomed Mat Res, 1992; 26:791-800. After the removal of bone wax, the amount of blood was measure with paper points in the same manner as mentioned above. The difference in weight before and after the blood collection was intended to represent the amount of blood seepage after the application of the hemostatic agent for hemostasis, and therefore, the hemostatic efficacy.

In the experimental group, each animal had two experimental sites, which were randomly assigned for two different test materials: cotton pellet with epinephrine (Racellet #3) and PU foam impregnated with epinephrine. Each experimental site served to test the hemostatic efficacy of the test materials. In the experimental site assigned to cotton pellet with epinephrine, one Racellet #3 cotton pellet was applied in the osseous defect with gentle compression for 2 minutes. In the contralateral site assigned to PU foam, a foam cube containing epinephrine, prepared as mentioned above, was applied to the osseous defect with gentle compression for 2 minutes in the same manner. For all the experimental sites, the hemostatic efficacy was measured with 15 pre-weighed paper points for 2 minutes after the removal of the hemostatic agent in the same manner as explained above.

The flaps were then repositioned and dosed with 4-0 absorbable vicryl sutures. Enrofloxacin (5 mg/kg) was administered to each animal for infection control and buprenorphine (0.01 mg/kg) for pain control.

### III. Preparation for Histological Evaluation

The animals were euthanized at seven weeks post-operatively using an overdose of ketamine (44 mg/kg) and pentobarbital (90 mg/kg). The mandibles were then dissected and split in the symphysis to include both the osseous defects and the adjacent bone. After removing the distal portion of the mandible, the specimens were placed in 10% buffered formalin for 2 weeks. The specimens were demineralized in 5% formic acid and then subsequently dehydrated in 30%, 70% and 100% alcohol. After imbedding in paraffin, serial mesiodistal sections of 5 μm thick were cut from the symphysis into the osseous defect. Ten to thirteen slides spanning the entire osseous defect were obtained. The slides were stained with H&E and evaluated under a light microscope.

### IV. Histological Examination of the Osseous Defects

The histological sections were examined by a blinded oral pathologist for the degree of inflammation and foreign body reaction. The severity of inflammation was scored on an ordinal scale from 1 to 4 based on the following descriptive scale used in a previous study conducted by Torabinejad et al., J Endod, 1997, 23:225-228:
1—None, no inflammatory cells.
2—Mild, few inflammatory cells.
3—Moderate, inflammatory cells do not obscure the normal tissues.
4—Severe, inflammatory cell replacing normal tissues.

The wound healing was scored on an ordinal scale from 1 to 5 based on the following descriptive scale used in the previous study conducted by Jeansonne et al, J Endod, 1993, 19:174:
1—Complete healing with surgical site filled with healthy cancellous bone.
2—Fibrosis with dense collagen, with or without early bone formation.
3—Granulation tissue filling the surgical site, with or without chronic inflammation. 4—Acute inflammation, with or without granulation tissue.
5—Abscess formation.

The degree of foreign body reaction was graded by counting the number of foci of foreign body giant cells in one field view at 40× under the microscope. The section with the maximum number of foci was selected for each specimen for comparison. The following descriptive scale was used to grade the degree of foreign body reaction:
0—None, 0 foci of foreign body giant cells (at 40× field view)
1—Mild, 1-5 foci of foreign body giant cells
2—Moderate, 6-10 foci of foreign body giant cells
3—Severe, >10 foci of foreign body giant cells

### V. Statistical Analysis

Statistical analysis was performed by using SPSS version 21 (IBM SPSS Statistics for Windows; IBM Corp, Armonk, N.Y.). The scores were analyzed by Pearson chi-square test and independent-samples Kruskal-Wallis test to determine whether statistically significant differences exist between and within the experimental groups and control groups. No adjustment was made for multiple comparisons. Statistical significance was determined at P<0.05.

### RESULTS

Experimental data from all 18 animals were included in this study for measurement of the hemostatic efficacy. One animal in the control group never regained consciousness from general anesthesia and was lost immediately after the experiment. One animal from the experimental group had histologic slides of poor quality and was excluded from the histological analysis. Therefore, specimens from 16 out of 18 animals were included for the histological analysis, comprising of 5 negative control sites, 5 positive control sites, 11 experimental sites for Racellet #3, and 11 experimental sites for PU foam cubes containing epinephrine.

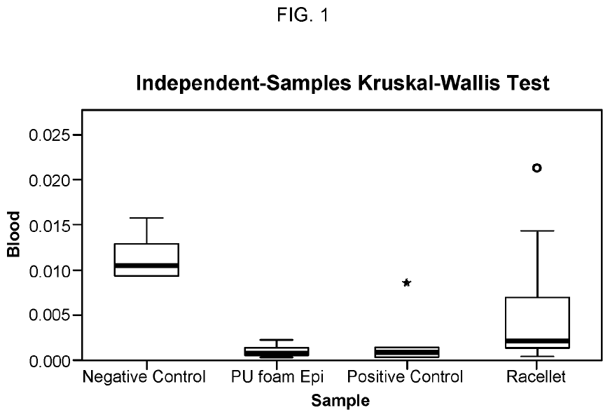

In regards to the hemostatic efficacy, complete hemostasis was achieved in all positive and experimental groups in the 2-minute period allowed for the application of the hemostatic agents. The results showed that the positive control group (bone wax) was significantly better in achieving hemostasis than the negative control group, which had no hemostatic intervention (P<0.05) (FIG. 1). PU foam with epinephrine was also significantly better in achieving hemostasis than both the negative control group (P<0.05) and the Racellet #3 group (P<0.05). Although Racellet #3 group was significantly better in achieving hemostasis than the negative control group, statistical analysis revealed no statistically significant difference between the two groups. In addition, although the positive control group was significantly better in achieving hemostasis than the Racellet #3 group, statistical analysis revealed no statistically significant difference between the two groups. There was no statistically significant difference in the hemostatic efficacy between the PU foam with epinephrine group and the positive control group.

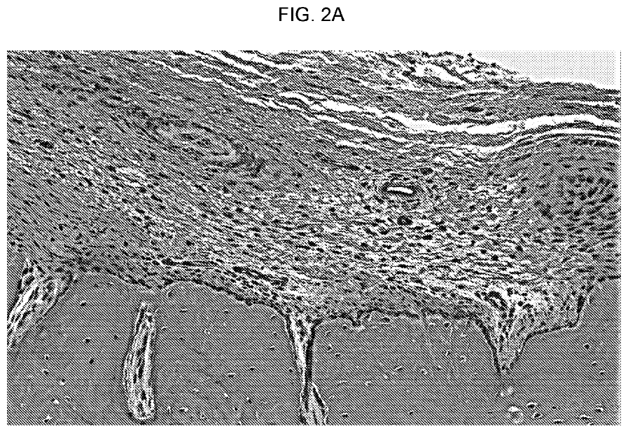

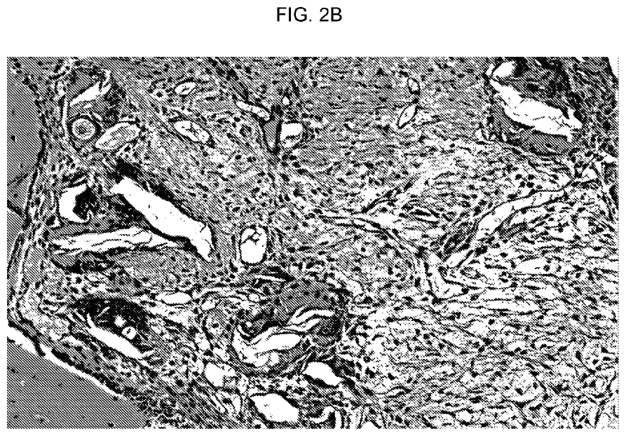

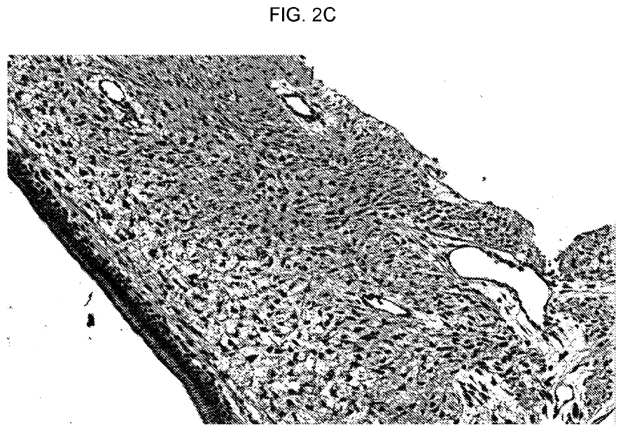

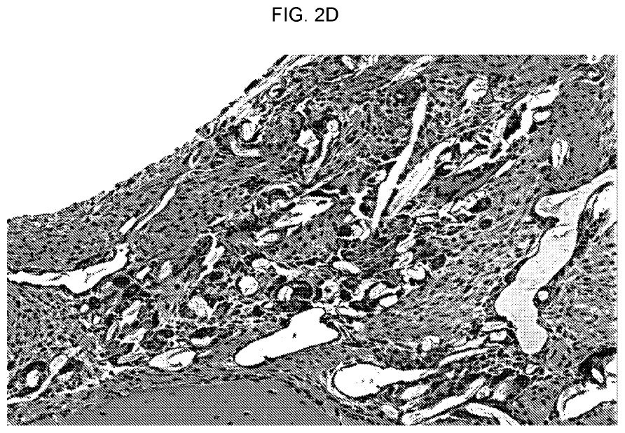

With respect to the severity of inflammation, all specimens exhibited no inflammation, corresponding to score 1 based on the descriptive scale in a previous study by Torabinejad et al, (21). See, FIGS. 2A-2D. With respect to wound healing, all specimens correlated with category 2 description based on the scale used in a previous study conducted by Jeansonne et al. (22)—fibrosis with dense collagen, with or without early bone formation. All of the specimens exhibited some bone formation.

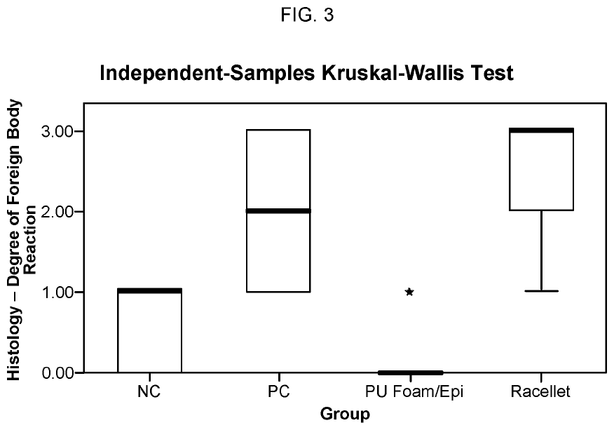

With respect to the degree of foreign body reaction, the results showed that PU foam containing epinephrine elicited markedly less foreign body reaction than Racellet #3, and this difference was statistically significant (P<0.05) (FIG. 3). In fact, only 2 out of 11 specimens showed foreign body giant cells in the PU foam group whereas 11 out of 11 specimens showed foreign body giant cells in the Racellet #3 group. Even for the two specimens that did show foreign body reaction in the PU foam group, they had a very mild response with only one small focus of foreign body giant cells. When compared to the negative control group, Racellet #3 elicited significantly more foreign body reaction (P<0.05) whereas PU foam with epinephrine showed no statistically significant difference. When compared to the positive control group, Racellet #3 showed no statistically significant difference in the foreign body reaction whereas PU foam with epinephrine showed significantly less foreign body reaction (P<0.05). Although the negative control group elicited significantly less foreign body reaction than the positive control group, statistical analysis revealed no statistically significant difference between the two groups.

### DISCUSSION

The results of our study indicate that PU foam impregnated with epinephrine was significantly better in achieving hemostasis than cotton pellet impregnated with epinephrine. This may be due in part to the above-mentioned hydrophilic property of the PU foam, which according to the manufacturer makes the foam capable of absorbing up to 15 times its weight of aqueous fluid. Clinically, there were noted differences in the handling properties of these two agents in both their application and removal. When using PU foam cubes, the sponge-like consistency of the material made it easy to conform to the osseous defect but the resilience of the material made it harder to pack into the defect, while cotton pellets were easily packed into the defect without a rebound effect. In the removal from the defect, PU foam cubes were very simple and straightforward and did not leave any visible residue behind. In contrast, the removal of cotton pellets resulted in cotton fibers sticking to the surfaces of the osseous defect, leaving behind very thin strands of fibers that were very difficult to visualize. For the purposes of this study, we did not attempt to curette out these thin strands of fibers remaining in the osseous defect.

In the second phase of this study, we compared the biocompatibility of cotton pellets and PU foam cubes by looking at the degree of inflammation, wound healing, and foreign body reaction histologically. The progression of wound healing occurs from hemostasis to inflammation, proliferation and maturation. See, e.g., Waldorf et al., Adv Dermatol., 1995, 10:77-96. Inflammation phase occurs during the first few days of wound healing and proliferative phase rolls in as angiogenesis, collagen deposition and granulation tissue formation happens. In this study, we could not find any inflammation in any of the specimens, regardless of the experimental groups. It can be speculated that the inflammation phase of wound healing had already been completed when the animals were sacrificed at 7-weeks post operatively in this study thus no signs of inflammation. It appears that wound healing had progressed onto the proliferative and remodeling phase in these animals as evidenced by the pattern seen in the observation of wound healing,

For the histological observation of wound healing, the in vivo animal model previously used by Jeansonne et al. to compare osseous wound healing of ferric sulfate versus controls in rabbits was used as the basis for this study. The results of this study showed that all specimens, regardless of the experimental groups, had a wound healing score of 2, which is fibrosis with dense collagen, with or without early bone formation. All of the specimens exhibited early bone formation with islands of bone surrounded by osteoblasts but no specimens had complete healing of the surgical site with complete bone formation. None of the specimens had abscess formation, which suggests that none of the surgical sites were infected.

Foreign body response is the non-specific immune response to implanted foreign materials (Anderson et al., Annu. Rev. Mater. Res. 2001, 31:81-110). The foreign body reaction is composed of macrophages and foreign body giant cells and it is the end-stage of the inflammatory and wound healing response following implantation of a foreign material Foreign body giant cells are the products of macrophage fusion, and are a hallmark of the foreign body reaction. When macrophages encounter a foreign object too large to be phagocytosed, they fuse to form larger foreign body giant cells composed of up to a few dozen individual macrophages. Giant cells secrete degradative agents such as superoxides and free radicals, causing localized damage to the foreign bodies. Eventually after the chronic inflammation, the foreign material becomes encapsulated in a dense layer of fibrotic connective tissue, which shields it from the immune system of the host and isolates it from the surrounding tissues. See, Anderson et al.; Luttikhuizen et al., Tissue Eng, 2006, 12:1955-1970.

In this study, biocompatibility of the two hemostatic agents was examined also by comparing the degree of foreign body reaction in the osseous defects. The results showed that PU foam with epinephrine elicited significantly less foreign body reaction than cotton pellets with epinephrine. All specimens in the cotton pellet with epinephrine group exhibited foreign body giant cells encircling the foreign body (cotton fibers) with 7 of 11 showing a severe degree of foreign body reaction. On the other hand, only 2 out of 11 specimens had foreign body giant cells in the PU foam with epinephrine group, and in both cases it was a very mild response with only one small focus of foreign body giant cells. This mild foreign body reaction in the PU foam group could have resulted from contamination during the experiment by fibers from paper points used to collect blood or microscopic residue from the PU foam. The result of the study suggests that when cotton pellets with epinephrine are used in osseous defects as a local hemostat, there is a good chance that small cotton fibers may be left behind even after the removal of the cotton pellet and these fibers can cause considerable foreign body reaction if not completely removed by the operator.

Moreover, the fact that PU foam with epinephrine showed statistically significantly less foreign body reaction than the positive control group (bone wax) but had no significant difference with the negative control group confirms that PU foam tested in this study is a good alternative material to cotton pellets and can be used as a local hemostatic agent in conjunction with epinephrine with minimal if any foreign body reaction. No significant difference between PU foam with epinephrine and the negative control group also suggests that tissue reaction to PU foam is comparable to that of no hemostatic intervention. The fact that cotton pellets with epinephrine was not significantly different from the positive control group (bone wax) and produced a significantly more intense foreign body reaction than the negative control indicate that the cotton fibers must be completely removed to avoid burdening the host with foreign body reaction which may lead to compromised or delayed healing.

In conclusion, epinephrine-impregnated PU foam cubes are a good alternative to epinephrine cotton pellets for local hemostasis in osseous defects created in the mandibles of guinea pigs. Epinephrine-impregnated PU foam cubes can display better hemostatic efficacy with minimal foreign body reaction due to its non-fibrous structure compared to epinephrine cotton pellets, which can elicit a severe foreign body reaction if cotton fibers that are readily retained in the surgical sites are not completely removed. As such, PU foam with epinephrine shows promise as an adjunct to the surgical armamentarium for endodontic surgery.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

What is claimed is:

1. A composition for promoting hemostasis, the composition comprising: a porous non-fibrous substrate impregnated with a hemostatic agent, wherein the porous non-fibrous substrate comprises a hydrophilic polyurethane foam.

2. The composition of claim 1, wherein the hemostatic agent is a vasoconstrictor or a chemical cauterizing agent.

3. The composition of claim 2, wherein the vasoconstrictor is epinephrine.

4. The composition of claim 3, wherein the composition comprises epinephrine in an amount from about 0.45 mg to about 0.75 mg.

5. The composition of claim 2, wherein the chemical cauterizing agent is ferric sulfate or calcium sulfate.

6.-11. (canceled)

12. A method of manufacturing a composition for promoting hemostasis, the method comprising:
applying an aqueous solution comprising a hemostatic agent onto a surface of a porous non-fibrous substrate comprising a hydrophilic polyurethane foam, wherein the aqueous solution is absorbed by the substrate.

13.-14. (canceled)

15. The method of claim 12, wherein the hemostatic agent is a vasoconstrictor or a chemical cauterizing agent.

16. The method of claim 12, wherein the hemostatic agent is a vasoconstrictor.

17. The method of claim 12, wherein the hemostatic agent is epinephrine.

18. The method of claim 12, wherein the aqueous solution further comprises an antioxidant.

19. The method of claim 18, wherein the antioxidant is sodium metabisulfate.

20.-25. (canceled)

26. The method of claim 12, wherein after the applying step, the method further comprises removing essentially all water from the porous non-fibrous substrate.

27. The method of claim 26, wherein the water is removed by air drying.

28. The method of claim 26, wherein the water is removed by lyophilization.

29. A method of promoting hemostasis in a subject in need thereof, the method comprising applying to an active bleeding site in the subject a composition containing a hydrophilic polyurethane foam impregnated with a hemostatic agent.

30. The method of claim 29, wherein the active bleeding site is a surgical site.

31. The method of claim 30, wherein the active bleeding site is an endodontic surgical site.

32. The method of claim 29, wherein the active bleeding site is a wound.

33. The method of claim 29, wherein the active bleeding site is a nosebleed.
